(19) United States
(12) Patent Application Publication
(10) Pub. No.: US 2009/0306388 A1
(43) Pub. Date: Dec. 10, 2009

(54) METHOD FOR SUBSTITUTED IH-IMIDAZO[4,5-C] PYRIDINES

(75) Inventors: Bernhard M. Zimmerman, Saint Paul, MN (US); Joseph F. Dellaria, JR., Saint Paul, MN (US); Luke T. Dressel, Saint Paul, MN (US); David T. Amos, Saint Paul, MN (US)

Correspondence Address: PFIZER INC.;PATENT DEPARTMENT, Bld 114 M/S 114, EASTERN POINT ROAD, GROTON, CT, 06340, US

(73) Assignee: Pfizer Inc.

(21) Appl. No.: 12/278,872

(22) PCT Filed: Feb. 9, 2007
(86) PCT No.: PCT/US07/03797

§ 371 Date: Mar. 24, 2009

Related U.S. Application Data
(60) Provisional application No. 60/743,437, filed on Mar. 8, 2006.

(30) Foreign Application Priority Data
Feb. 10, 2006 (US) PCT/US2006/004737

(51) Int. Cl. C07D 471/02

(52) U.S. Cl. 546/118

(57) ABSTRACT

Methods for preparing compounds of the Formulas IV and I are disclosed. The methods include combining a compound of the Formula II: with a benzylamine of the Formula III: in the presence of an acid to provide a 1H-imidazo[4,5-c]pyridine compound of the Formula IV.

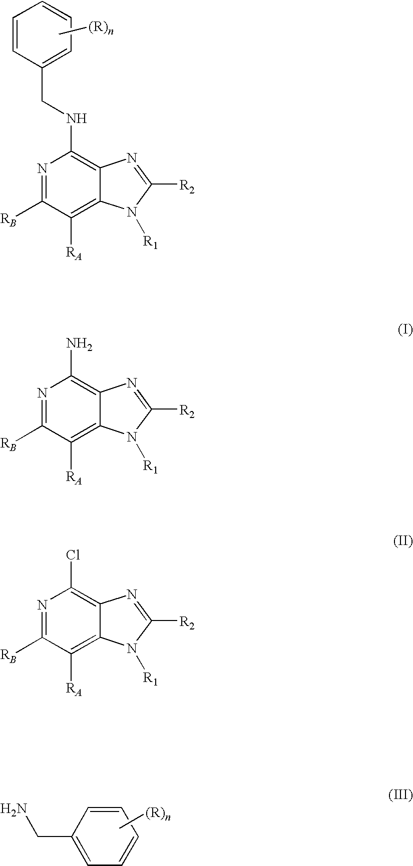

### CROSS REFERENCE TO RELATED APPLICATIONS

The present invention claims priority to U.S. Provisional Application Ser. No. 60/743,437, filed Mar. 8, 2006, and to International Application No. PCT/US2006/004737, filed Feb. 10, 2006, both of which are incorporated herein by reference.

### BACKGROUND

Certain compounds have been found to be useful as immune response modifiers (IRMs), rendering them useful in the treatment of a variety of disorders. However, there continues to be interest in and a need for compounds that have the ability to modulate the immune response, by induction of cytokine biosynthesis or other mechanisms. Thus, there is a need for methods and intermediates for making such compounds.

### SUMMARY

It has now been found that certain 1H-imidazo[4,5-c]pyridines which are substituted at the 4-position, or pharmaceutically acceptable salts thereof, can be prepared by a method comprising:

combining a compound of the Formula II:

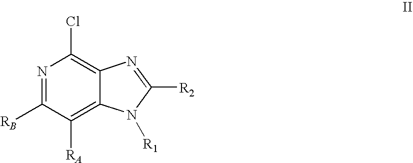

with a benzylamine of the Formula III:

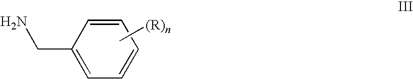

in the presence of an acid to provide a 1H-imidazo[4,5-c]pyridine compound of the Formula IV:

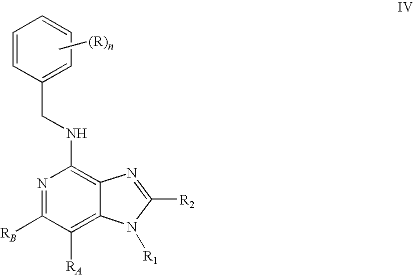

or a pharmaceutically acceptable salt thereof; wherein R, n, R₁, R₂, R_{A}, and R_{B} are defined below.

Compounds and salts of Formula IV are useful for making immune response modifying compounds of the following Formula I:

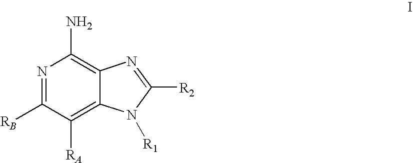

or pharmaceutically acceptable salts thereof; wherein R₁, R₂, R_{A}, and R_{B} are defined below. The compounds and salts of Formula I are known to be useful as immune response modifiers due to their ability to induce or inhibit cytokine biosynthesis (e.g., induces or inhibits the biosynthesis of at least one cytokine) and otherwise modulate the immune response when administered to animals. This makes these compounds and salts useful in the treatment of a variety of conditions such as viral diseases and tumors that are responsive to such changes in the immune response.

In one embodiment, there is provided a method that includes:

combining a compound of the Formula II:

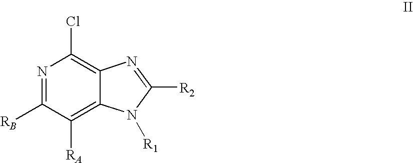

with a benzylamine of the Formula III:

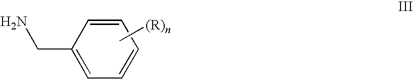

in the presence of an acid to provide a 1H-imidazo[4,5-c]pyridine compound of the Formula IV:

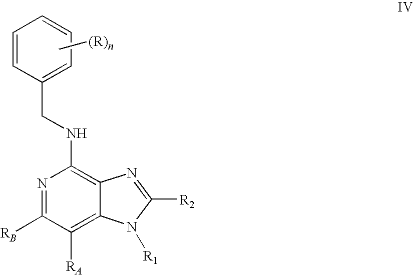

or a pharmaceutically acceptable salt thereof; wherein R, n, R₁, R₂, R_{A}, and R_{B} are defined below; and

solvolyzing the compound or salt of Formula IV or hydrogenolyzing the compound or salt of Formula IV to provide a 1H-imidazo[4,5-c]pyridin-4-amine compound of the Formula I:

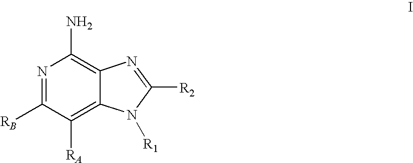

or a pharmaceutically acceptable salt thereof; wherein R₁, R₂, R_{A}, and R_{B} are defined below.

As used herein, “a”, “an”, “the”, “at least one”, and “one or more” are used interchangeably.

The terms “comprises” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS OF THE INVENTION

The present invention provides methods for preparing certain 1H-imidazo[4,5-c]pyridines which are substituted at the 4-position. In one embodiment, there is provided a method comprising:

combining a compound of the Formula II:

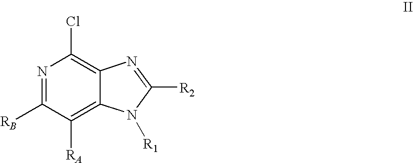

with a benzylamine of the Formula III:

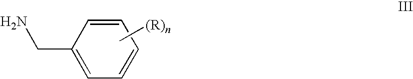

in the presence of an acid to provide a 1H-imidazo[4,5-c]pyridine compound of the Formula IV:

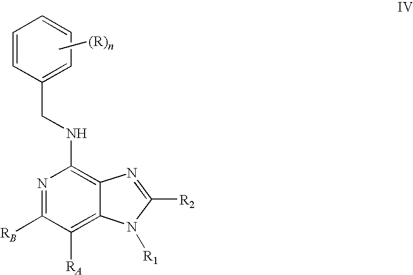

or a pharmaceutically acceptable salt thereof;
wherein in the above Formulas III and IV:

each R is independently selected from the group consisting of alkyl, alkoxy, aryloxy, alkylamino, dialkylamino, arylamino, alkylthio, arylthio, and halogen; and

n is 0, 1, 2, or 3; and

wherein in the above Formulas II and IV:

R_{A} and R_{B} are independently selected from the group consisting of:
hydrogen,
halogen,
alkyl,
alkenyl,
alkoxy,
alkylthio, and
—N(R₉)₂;

R₁ is selected from the group consisting of:
—R₄,
—X—R₄,
—X—Y—R₄,
—X—Y—X—Y—R₄, and
—X—R₅,

R₂ is selected from the group consisting of:
—R₄,
—X—R₄,
—X—Y—R₄, and
—X—R₅;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

Y is selected from the group consisting of:
—O—,
—S(O)₀₋₂—,
—S(O)₂—N(R₈)—,
—C(R₆)—,
—C(R₆)—O—,
—O—C(R₆)—,
—O—C(O)—O—,
—N(R₆)-Q-,
—C(R₆)—N(R₈)—,
—O—C(R₆)—N(R₈)—,
—C(R₆)—N(OR₈)—,
—O—N(R₈)-Q-,
—O—N═C(R₄)—,
—C(—N—O—R₈)—,
—CH(—N(—O—R₈)-Q-R₄)—,

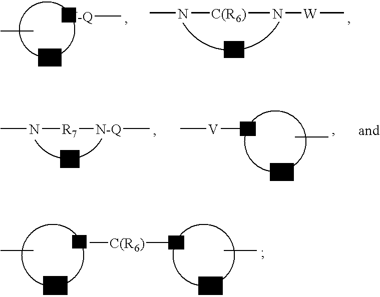

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, heterocyclyl, and heterocyclylalkylenyl; wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo; and wherein the heterocyclylalkylenyl group is optionally substituted by one or more alkyl groups;

R₅ is selected from the group consisting of:

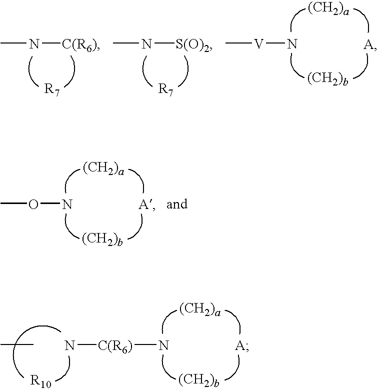

R₆ is selected from the group consisting of ═O and ═S;

R₇ is C₂₋₇ alkylene;

R₈ is selected from the group consisting of hydrogen, alkyl, alkoxyalkylenyl, hydroxyalkylenyl, arylalkylenyl, and heteroarylalkylenyl;

R₉ is selected from the group consisting of hydrogen and alkyl;

R₁₀ is C₃₋₈ alkylene;

A is selected from the group consisting of —CH₂—, —O—, —C(O)—, —S(O)₀₋₂—, and —N(Q-R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7;

with the proviso that R₁ is other than (4-hydroxytetrahydro-2H-pyran-4-yl)methyl.

For certain embodiments, the compound of Formula II is combined with at least one equivalent of the compound of Formula III.

For certain embodiments, the compound of Formula II is combined with at least two equivalents of the compound of Formula III.

For certain embodiments, the compound of Formula II is combined with at least three equivalents of the compound of Formula III.

For certain embodiments, the compound of Formula II is combined with at least four equivalents of the compound of Formula III.

For certain embodiments, the compound of Formula II is combined with at least five equivalents of the compound of Formula III.

For certain embodiments, including any one of the above embodiments, the compound of Formula II is combined with an amount of the compound of Formula III sufficient to act as solvent.

For certain embodiments, including any one of the above embodiments, the compound of Formula II is combined with up to twenty equivalents of the compound of Formula III.

For certain embodiments, including any one of the above embodiments, the compound of Formula II is combined with up to fifteen equivalents of the compound of Formula III.

For certain embodiments, including any one of the above embodiments, the compound of Formula II is combined with up to six equivalents of the compound of Formula III.

For certain embodiments, including any one of the above embodiments, the compound of Formula II is combined with up to five equivalents of the compound of Formula III.

For certain embodiments, including any one of the above embodiments, the compound of Formula III is selected from the group consisting of benzylamine, 4-methoxybenzylamine, 2,4-dimethoxybenzylamine, and 3,4-dimethoxybenzylamine.

For certain embodiments, including any one of the above embodiments, the pKa of the acid is up to 9.3. For certain of these embodiments, the pKa of the acid is up to 8. For certain of these embodiments, the pKa of the acid is up to 7. For certain of these embodiments, the pKa of the acid is up to 5.

For certain embodiments, including any one of the above embodiments, the number of equivalents of acid present is equal to or less than the number of equivalents of the compound of Formula III. For certain of these embodiments, the number of equivalents of acid present is up to 0.75 of the number of equivalents of the compound of Formula III. For certain of these embodiments, the number of equivalents of acid present is up to 0.5 of the number of equivalents of the compound of Formula III. For certain of these embodiments, the number of equivalents of acid present is up to 0.4 of the number of equivalents of the compound of Formula III. For certain of these embodiments, the number of equivalents of acid present is up to 0.25 of the number of equivalents of the compound of Formula III. For certain of these embodiments, the number of equivalents of acid present is up to 0.15 of the number of equivalents of the compound of Formula III.

For certain embodiments, including any one of the above embodiments, a solvent is present. For certain of these embodiments, the solvent is less nucleophilic than the compound of Formula III. For certain of these embodiments, the solvent is a polar solvent. For certain of these embodiments, the polar solvent is a protic solvent. For certain of these embodiments, the solvent is selected from the group consisting of methanol, ethanol, 1-propanol, 2-propanol, 1-butanol, 2-butanol, 2-methyl-1-propanol, 2-methyl-2-propanol, 2,2,2-trifluoroethanol, ammonium hydroxide, water, and mixtures thereof. For certain of these embodiments, the solvent is 2,2,2-trifluoroethanol. Alternatively, for certain of these embodiments, the polar solvent is an aprotic solvent. For certain of these embodiments, the solvent is selected from the group consisting of acetonitrile, acetone, 1-methyl-2-pyrrolidinone, dimethyl sulfoxide, pyridine, N,N-dimethylformamide, 1,3-dimethyl-3,4,5,6-tetrahydro-2(1H)-pyrimidinone, and mixtures thereof.

For certain embodiments, including any one of the above embodiments, the temperature of the combined compounds in the presence of the acid is at least room temperature. For certain of these embodiments, the temperature is an elevated temperature. For certain of these embodiments, the temperature is at least 60° C. For certain of these embodiments, the temperature is at least 80° C. For certain of these embodiments, the temperature is at least 120° C. For certain of these embodiments, the temperature is up to 250° C. For certain of these embodiments, the temperature is up to 180° C. For certain of these embodiments, the temperature is up to 150° C.

For certain embodiments, including any one of the above embodiments which includes a solvent, the temperature of the combined compounds in the presence of the acid is at the reflux temperature of the solvent.

For certain embodiments, including any one of the above embodiments which includes an elevated temperature, the temperature of the combined compounds in the presence of the acid is provided by exposure to microwaves. For certain of these embodiments, the combined compounds in the presence of the acid are in a sealed container.

For certain embodiments, including any one of the above embodiments which includes an elevated temperature, the temperature of the combined compounds in the presence of the acid is provided by external heating, such as, for example, by contact, convective, or radiative heating with a heat source.

For certain embodiments, including any one of the above embodiments, the time required for the compound or salt of Formula IV to be provided in a yield of about 50% or more is up to 1 hour. For certain of these embodiments, the time is 30 minutes.

For certain embodiments, the above method or any one of its above embodiments further comprises a step selected from the group consisting of solvolyzing the compound or salt of Formula IV and hydrogenolyzing the compound or salt of Formula IV to provide a 1H-imidazo[4,5-c]pyridin-4-amine compound of the Formula I:

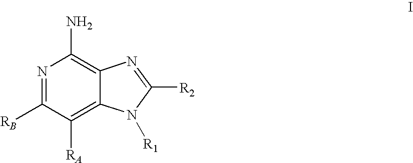

or a pharmaceutically acceptable salt thereof, wherein R₁, R₂, R_{A}, and R_{B} are defined as in Formula IV above. For certain of these embodiments, the step is solvolyzing the compound or salt of Formula IV to provide a 1H-imidazo[4,5-c]pyridin-4-amine compound of the Formula I. For certain of these embodiments, the step of solvolyzing is carried out under acidic conditions. For certain of these embodiments the step of solvolyzing is carried out in an excess of an acid, such as, for example, trifluoroacetic acid, sulfuric acid, or trifluorosulfonic acid. For certain of these embodiments, the acid is trifluoroacetic acid. Alternatively, for certain of these embodiments, the step is hydrogenolyzing the compound or salt of Formula IV to provide a 1H-imidazo[4,5-c]pyridin-4-amine compound of the Formula I. For certain of these embodiments, the step of hydrogenolyzing is carried out with a heterogeneous catalyst. For certain of these embodiments, the heterogeneous catalyst is palladium on carbon. For certain of these embodiments, the step of hydrogenolyzing is carried out with palladium on carbon and ammonium formate.

For certain embodiments, including any one of the above embodiments, the method further comprises the steps of providing a compound of the Formula XII:

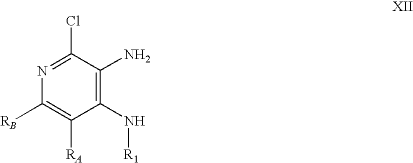

wherein R_{A}, R_{B}, and R₁ are defined as in Formula IV above;

and reacting the compound of the Formula XII with a carboxylic acid of the formula R₂CO₂H; an equivalent thereof selected from the corresponding acyl halide, R₂C(O-alkyl)₃, and R₂C(O-alkyl)₂(O—C(O)-alkyl); or a mixture thereof, wherein R₂ is defined as in Formula IV above, and each alkyl contains 1 to 8 carbon atoms, to provide a compound of the Formula II:

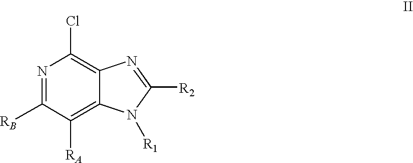

wherein R_{A}, R_{B}, R₁, and R₂ are defined as in Formula IV above.

For certain embodiments, including any one of the above embodiments, the method further comprises the steps of providing a compound of the Formula XI:

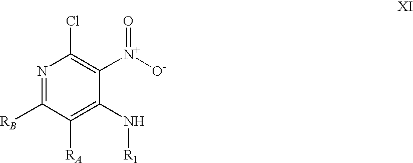

wherein R_{A}, R_{B}, and R₁ are defined as in Formula IV above;

and reducing the compound of Formula XI to provide a compound of the Formula XII described above.

For certain embodiments, including any one of the above embodiments, the method further comprises the steps of providing a compound of the Formula X:

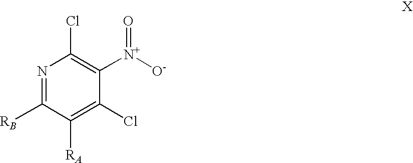

wherein R_{A} and R_{B} are defined as in Formula IV above;

and reacting the compound of Formula X with an amine of Formula R₁—NH₂, wherein R₁ is defined as in Formula IV above, to provide a compound of Formula XI described above.

For certain embodiments, including any one of the above embodiments, R_{A} and R_{B} are each independently selected from the group consisting of hydrogen, halogen, alkyl, alkenyl, alkoxy, alkylthio, and —N(R₉)₂.

For certain embodiments, including any one of the above embodiments, R_{A} is hydrogen or alkyl, and R_{B} is alkyl. For certain of these embodiments, R_{A} and R_{B} are both methyl.

For certain embodiments, including any one of the above embodiments, R₁ is selected from the group consisting of —R₄, —X—R₄, —X—Y—R₄, —X—Y—X—Y—R₄, and —X—R₅; wherein when R₁ is —R₄ or —X—R₄, then X is selected from the group consisting of straight chain or branched chain alkylene, alkenylene, alkynylene, arylene, heteroarylene, and heterocyclylene wherein the straight chain or branched chain alkylene, alkenylene, and alkynylene groups can be optionally interrupted or terminated by arylene, heteroarylene or heterocyclylene and optionally interrupted by one or more —O— groups; and R₄ is selected from the group consisting of hydrogen, straight chain or branched chain alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, heterocyclyl, and heterocyclylalkylenyl; wherein the straight chain or branched chain alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of straight chain or branched chain alkyl, alkenyl, alkynyl, and heterocyclyl, oxo; and wherein the heterocyclylalkylenyl group is optionally substituted by one or more alkyl groups. For certain of these embodiments, R₁ is —R₄ or —X—R₄. For certain of these embodiments, R₁ is selected from the group consisting of alkyl, arylalkylenyl, aryloxyalkylenyl, hydroxyalkyl, dihydroxyalkyl, and alkoxyalkylenyl, wherein alkyl and alkylenyl are straight chain or branched chain. For certain of these embodiments, R₁ is selected from the group consisting of 2-hydroxy-2-methylpropyl, 2-methylpropyl, propyl, ethyl, methyl, 2,3-dihydroxypropyl, 3-isopropoxypropyl, and 2-phenoxyethyl. Alternatively, for certain of these embodiments where R₁ is —R₄ or —X—R₄, R₁ is heterocyclylalkylenyl which is optionally substituted by one or more alkyl groups. For certain of these embodiments, R₁ is tetrahydro-2H-pyran-4-ylmethyl or (2,2-dimethyl-1,3-dioxolan-4-yl)methyl.

For certain embodiments, including any one of the above embodiments, R₁ is —X—Y—R₄ or —X—R₅ except where the definition of R₁ does not include this definition. For certain of these embodiments, X is alkylene; Y is —N(R₈)—C(R₆)—, —N(R₈)—S(O)₂—,

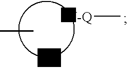

—N(R₈)—C(O)—N(R₈)—, —C(R₆)—N(R₈)—, —C(R₆)—O—, or R₄ is alkyl, aryl, or heteroaryl; and R₅ is

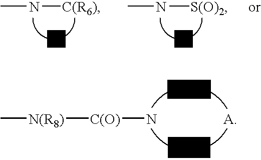

For certain of these embodiments, R₁ is 4-[(methylsulfonyl)amino]butyl, 2-methyl-2-[(methylsulfonyl)amino]propyl, 2-(acetylamino)-2-methylpropyl, 2-{[(isopropylamino)carbonyl]amino}-2-methylpropyl, 4-{[(isopropylamino)carbonyl]amino}butyl, or 4-(1,1-dioxidoisothiazolidin-2-yl)butyl.

For certain embodiments, including any one of the above embodiments except where the definition of R₁ does not include the following definition, R₁ is selected from the group consisting of alkyl, arylalkylenyl, aryloxyalkylenyl, hydroxyalkyl, dihydroxyalkyl, alkoxyalkylenyl, alkylsulfonylalkylenyl, —X—Y—R₄, —X—R₅, and heterocyclylalkylenyl; wherein the heterocyclyl of the heterocyclylalkylenyl group is optionally substituted by one or more alkyl groups; wherein X is alkylene; Y is —N(R₈)—C(R₆)—, —N(R₈)—S(O)₂—, —N(R₈)—C(O)—N(R₈)—, —C(R₆)—N(R₈)—, —C(R₆)—O—, or

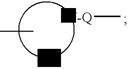

R₄ is alkyl, aryl, or heteroaryl; and R₅ is

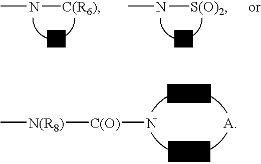

For certain of these embodiments, alkyl and alkylenyl in alkyl, arylalkylenyl, aryloxyalkylenyl, hydroxyalkyl, dihydroxyalkyl, alkoxyalkylenyl, and alkylsulfonylalkylenyl are straight chain or branched chain. For certain of these embodiments, R₁ is selected from the group consisting of 2-hydroxy-2-methylpropyl, 2-methylpropyl, propyl, ethyl, methyl, 2,3-dihydroxypropyl, 3-isopropoxypropyl, 2-phenoxyethyl, 4-[(methylsulfonyl)amino]butyl, 2-methyl-2-[(methylsulfonyl)amino]propyl, 2-(acetylamino)-2-methylpropyl, 2-{[(isopropylamino)carbonyl]amino}-2-methylpropyl, 4-{[(isopropylamino)carbonyl]amino}butyl, 4-(1,1-dioxidoisothiazolidin-2-yl)butyl, tetrahydro-2H-pyran-4-ylmethyl, and (2,2-dimethyl-1,3-dioxolan-4-yl)methyl.

For certain embodiments, including any one of the above embodiments except where the definition of R₁ does not include the following definition, R₁ is —X—Y—R₄. For certain of these embodiments, X is C₂₋₄ alkylene, and Y is —N(R₈)-Q-. For certain of these embodiments, —X—Y—R₄ is selected from the group consisting of 4-[(methylsulfonyl)amino]butyl, 2-methyl-2-[(methylsulfonyl)amino]propyl, 2-(acetylamino)-2-methylpropyl, 2-{[(isopropylamino)carbonyl]amino}-2-methylpropyl, and 4-{[(isopropylamino)carbonyl]amino}butyl.

For certain embodiments, including any one of the above embodiments except where the definition of R₁ does not include the following definition, R₁ is —X—R₅. For certain of these embodiments, —X—R₅ is 4-(1,1-dioxidoisothiazolidin-2-yl)butyl or 4-[(morpholin-4-ylcarbonyl)amino]butyl.

For certain embodiments, including any one of the above embodiments, R₂ is selected from the group consisting of —R₄, —X—R₄, —X—Y—R₄, and —X—R₅.

For certain embodiments, including any one of the above embodiments, R₂ is —R₄.

For certain embodiments, including any one of the above embodiments, R₂ is selected from the group consisting of hydrogen, alkyl, alkoxyalkylenyl, and hydroxyalkylenyl. For certain of these embodiments, R₂ is selected from the group consisting of hydrogen, methyl, ethyl, propyl, butyl, ethoxymethyl, methoxymethyl, 2-methoxyethyl, hydroxymethyl, and 2-hydroxyethyl.

For certain embodiments, R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, heterocyclyl, and heterocyclylalkylenyl; wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo; and wherein the heterocyclylalkylenyl group is optionally substituted by one or more alkyl groups.

For certain embodiments, R₄ is straight chain or branched alkyl, aryl, or heteroaryl.

For certain of these embodiments, R₄ is straight chain or branched chain alkyl optionally substituted by hydroxy. For certain of these embodiments, R₄ is 2-methylpropyl or 2-hydroxy-2-methylpropyl.

For certain embodiments, including any one of the above embodiments wherein R₄ is present in —X—Y—R₄, R₄ is C₁₋₄ alkyl. For certain of these embodiments, R₄ is methyl.

For certain embodiments, R₅ is selected from the group consisting of:

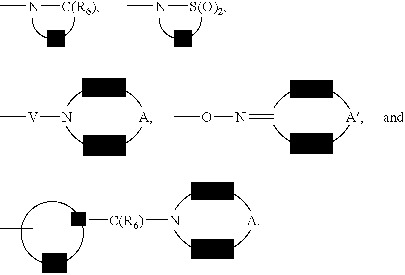

For certain of these embodiments, R₅ is

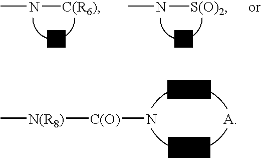

For certain of these embodiments, R₅ is

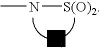

For certain of these embodiments, R₅ is

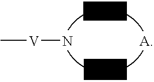

For certain of these embodiments, V is —NH—C(O)—. For certain of these embodiments, A is —O—. For certain of these embodiments, a and b are each 2.

For certain embodiments, each R is independently selected from the group consisting of alkyl, alkoxy, aryloxy, alkylamino, dialkylamino, arylamino, alkylthio, arylthio, and halogen. For certain of these embodiments, each R is alkoxy.

For certain embodiments, n is 0, 1, 2, or 3. For certain of these embodiments, n is 2. For certain of these embodiments, n is 1. For certain of these embodiments, n is 0.

For certain embodiments, X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups. For certain of these embodiments, alkylene is straight chain or branched chain. For certain of these embodiments, X is straight chain or branched chain alkylene. For certain of these embodiments, X is straight chain or branched chain C₂₋₆ alkylene. For certain of these embodiments, X is straight chain or branched chain C₂₋₄ alkylene.

For certain embodiments, Y is selected from the group consisting of —O—, —S(O)₀₋₂—, —S(O)₂—N(R₈)—, —C(R₆)—, —C(R₆)—O—, —O—C(R₆)—, —O—C(O)—O—, —N(R₈)-Q-, —C(R₆)—N(R₈)—, —O—C(R₆)—N(R₈)—, —C(R₆)—N(OR₈)—, —O—N(R₈)-Q-, —O—N═C(R₄)—, —C(═N—O—R₈)—, —CH(—N(—O—R₈)-Q-R₄)—,

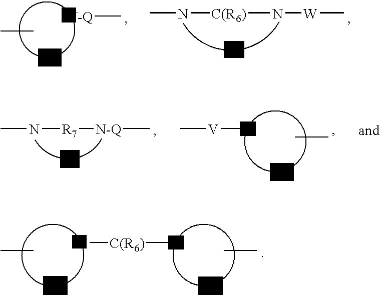

For certain embodiments, Y is —N(R₈)—C(R₆)—, —N(R₈)—S(O)₂—, —N(R₈)—C(O)—N(R₈)—, —C(R₆)—N(R₈)—, —C(R₆)—O—, or

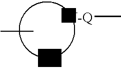

For certain embodiments, Y is —N(R₈)-Q-. For certain of these embodiments, Q is —C(O)—, —S(O)₂—, or —C(O)—NH—.

As used herein, a polar solvent is a solvent that facilitates the development of charge separation during the reaction, for example, the reaction of the benzylamine of Formula III with the compound of Formula II.

The pKa, as used herein, is measured in a water based system.

Unless otherwise specified, as used herein, the terms “alkyl”, “alkenyl”, “alkynyl” and the prefix “alk-” are inclusive of both straight chain and branched chain groups and of cyclic groups, e.g., cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclobutyl, cyclobutylmethyl, cyclopentyl, cyclopentylmethyl, cyclohexyl, cyclohexylmethyl, adamantyl, and substituted and unsubstituted bornyl, norbornyl, and norbornenyl.

Unless otherwise specified, “alkylene”, “alkenylene”, and “alkynylene” refer to a divalent form of the “alkyl”, “alkenyl”, and “alkynyl” groups defined above. The terms, “alkylenyl”, “alkenylenyl”, and “alkynylenyl” are used when “alkylene”, “alkenylene”, and “alkynylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-.” Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2-12 carbon atoms, 1-3 rings, 1-4 heteroatoms, and O, S, and N as the heteroatoms. Exemplary heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2-12 carbon atoms, 1-3 rings, 1-4 heteroatoms, and O, S, and N as the heteroatoms. Exemplary heterocyclyl groups include pyrrolidinyl, tetrahydrofuranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl (azepanyl), 1,4-oxazepanyl, homopiperazinyl (diazepanyl), 1,3-dioxolanyl, aziridinyl, azetidinyl, dihydroisoquinolin-(1H)-yl, octahydroisoquinolin-(1)-yl, dihydroquinolin-(2H)-yl, octahydroquinolin-(2H)-yl, dihydro-1H-imidazolyl, 3-azabicyclo[3.2.2]non-3-yl, and the like.

The term “heterocyclyl” includes bicyclic and tricyclic heterocyclic ring systems. Such ring systems include fused and/or bridged rings and spiro rings. Fused rings can include, in addition to a saturated or partially saturated ring, an aromatic ring, for example, a benzene ring. Spiro rings include two rings joined by one spiro atom and three rings joined by two spiro atoms.

When “heterocyclyl” contains a nitrogen atom, the point of attachment of the heterocyclyl group may be the nitrogen atom.

The terms “arylene”, “heteroarylene”, and “heterocyclylene” refer to a divalent form of the “aryl”, “heteroaryl”, and “heterocyclyl” groups defined above. The terms, “arylenyl”, “heteroarylenyl”, and “heterocyclylenyl” are used when “arylene”, “heteroarylene”, and “heterocyclylene”, respectively, are substituted. For example, an alkylarylenyl group comprises an arylene moiety to which an alkyl group is attached.

When a group (or substituent or variable) is present more than once in any Formula described herein, each group (or substituent or variable) is independently selected, whether explicitly stated or not. For example, when more than one Y group is present, then each Y group is independently selected. In another example, in the formula —N(R₉)₂, each R₉ group is independently selected.

The invention is inclusive of the methods with the compounds described herein in any of their pharmaceutically acceptable forms, including isomers (e.g., diastereomers and enantiomers), salts, solvates, polymorphs, prodrugs, and the like. In particular, if a compound is optically active, the methods of the invention specifically include each of the compound's enantiomers as well as racemic mixtures of the enantiomers. It should be understood that the term “compound” includes any or all of such forms, whether explicitly stated or not (although at times, “salts” are explicitly stated).

### Preparation of the Compounds

More specific details of the reactions described herein are discussed in the context of the following Reaction Scheme.

Some embodiments of the invention are described below in the Reaction Scheme. For more detailed description of the individual reaction steps, see the EXAMPLES section below. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, N.Y., (1967-1999 ed.); Alan R. Katritsky, Otto Meth-Cohn, Charles W. Rees, Comprehensive Organic Functional Group Transformations, v. 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

Although specific starting materials and reagents are depicted in the reaction scheme and discussed below, other starting materials and reagents known to those skilled in the art can be substituted to provide a variety of derivatives and/or reaction conditions. In addition, many of the methods described below can be further elaborated in light of this disclosure using conventional methods well known to those skilled in the art.

In carrying out methods of the invention it may sometimes be necessary to protect a particular functionality while reacting other functional groups on an intermediate. The need for such protection will vary depending on the nature of the particular functional group and the conditions of the reaction step. Suitable amino protecting groups include acetyl, trifluoroacetyl, tert-butoxycarbonyl (Boc), benzyloxycarbonyl (CBZ), and 9-fluorenylmethoxycarbonyl (Fmoc). Suitable hydroxy protecting groups include acetyl and silyl groups such as the tert-butyl dimethylsilyl group. For a general description of protecting groups and their use, see T. W. Greene and P. G. M. Wuts, Protective Groups in Organic Synthesis, John Wiley & Sons, New York, USA, 1991.

Conventional methods and techniques of separation and purification can be used to isolate compounds shown in the Reaction Scheme below. Such techniques may include, for example, all types of chromatography (high performance liquid chromatography (HPLC), column chromatography using common absorbents such as silica gel, and thin layer chromatography), recrystallization, and differential (i.e., liquid-liquid) extraction techniques.

Methods of the invention are shown in the Reaction Scheme below wherein R, R₁, R₂, R_{A}, R_{B}, and n are as defined above.

In step (1) of the Reaction Scheme, a 2,4-dichloro-3-nitropyridine of Formula X is reacted with an amine of Formula R₁—NH₂ to provide a 2-chloro-3-nitropyridin-4-amine of Formula XI. The reaction can be carried out by adding the amine of Formula R₁—NH₂ to a solution of the compound of Formula X in a suitable solvent such as N,N-dimethylformamide (DMF) in the presence of a tertiary amine such as triethylamine. The reaction can be carried out at ambient temperature or at a sub-ambient temperature such as, for example, 0° C. Some 2,4-dichloro-3-nitropyridines of Formula X are known; others can be prepared using known synthetic methods. See, for example, U.S. Pat. No. 6,525,064 (Dellaria) and the references cited therein. Numerous primary amines of Formula R₁—NH₂, or the salts thereof, are commercially available; others can be prepared using known synthetic methods. See, for example, the methods in U.S. Pat. No. 6,451,810 (Coleman), U.S. Pat. No.6,660,747 (Crooks), U.S. Pat. No.6,683,088 (Crooks), and U.S. Pat. No. 6,656,938 (Crooks); U.S. Patent Application Publication No. 2004/0147543 (Hays et al.); and International Publication No. WO2005/051317 (Krepski).

In step (2) of the Reaction Scheme, a 2-chloro-3-nitropyridin-4-amine of Formula XI is reduced to provide a 2-chloropyridine-3,4-diamine of Formula XII. The reduction can be carried out by a number of conventional methods. For example, the reduction can be carried out by hydrogenation using a heterogeneous hydrogenation catalyst such as platinum on carbon. The hydrogenation can be conveniently carried out in a Parr apparatus in a suitable solvent such as ethyl acetate at ambient temperature. The reduction can also be carried out using nickel boride, prepared in situ from sodium borohydride and nickel(II) chloride. The nickel boride reduction can be carried out by adding a solution of a compound of Formula XI in a suitable solvent or solvent mixture such as dichloromethane/methanol to a mixture of excess sodium borohydride and catalytic or stoichiometric nickel(II) chloride in methanol. The reaction can be carried out at room temperature. Alternatively the reduction can be carried out using a one- or two-phase sodium dithionite reduction. The sodium dithionite reduction can be conveniently carried out using the conditions described by Park, K. K.; Oh, C. H.; and Joung, W. K.; Tetrahedron Lett., 34, pp. 7445-7446 (1993) by adding sodium dithionite to a compound of Formula XI in a mixture of dichloromethane and water at ambient temperature in the presence of potassium carbonate and ethyl viologen dibromide, ethyl viologen diiodide, or 1,1′-di-n-octyl-4,4′-bipyridinium dibromide. Many compounds of Formula XI are known; see, for example, U.S. Pat. No. 6,525,064 (Dellaria), U.S. Pat. No. 6,545,016 (Dellaria), U.S. Pat. No. 6,545,017 (Dellaria), and U.S. Pat. No. 6,797,718 (Dellaria) and International Publication Numbers WO 2005/018551 (Kshirsagar), WO 2005/018556 (Kshirsagar), WO 2005/048933 (Kshirsagar), and WO 2005/051317 (Krepski).

In step (3) of the Reaction Scheme, a 2-chloropyridine-3,4-diamine of Formula XII is reacted with a carboxylic acid or an equivalent thereof to provide a 4-chloro-1H-imidazo[4,5-c]pyridine of Formula II. Suitable carboxylic acid equivalents include orthoesters of Formula R₂C(O-alkyl)₃, 1,1-dialkoxyalkyl alkanoates of Formula R₂C(O-alkyl)₂(O—C(O)-alkyl), and acid chlorides of Formula R₂C(O)Cl. The selection of the carboxylic acid equivalent is determined by the desired substituent at R₂. For example, triethyl orthoformate will provide a compound where R₂ is hydrogen, and trimethyl orthovalerate will provide a compound where R₂ is a butyl group. The reaction can be carried out by adding the carboxylic acid equivalent to a compound of Formula XII in a suitable solvent such as toluene. Optionally, catalytic pyridine hydrochloride can be added. The reaction is carried out at a temperature high enough to drive off alcohol or water formed during the reaction. Conveniently, a Dean-Stark trap can be used to collect the volatiles.

Alternatively, step (3) can be carried out in two steps when an acid chloride of Formula R₂C(O)Cl is used as the carboxylic acid equivalent. Part (i) of step (3) can be carried out by adding the acid chloride to a solution of a compound of Formula XII in a suitable solvent such as dichloromethane or acetonitrile to afford an amide. Optionally, a tertiary amine such as triethylamine, pyridine, or 4-dimethylaminopyridine can be added. The reaction can be carried out at room temperature or at a sub-ambient temperature such as, for example, 0° C. The amide product can be isolated and optionally purified using conventional techniques. Part (ii) of step (3) involves heating the amide prepared in part (i) to provide a 4-chloro-1H-imidazo[4,5-c]pyridine of Formula II. The reaction can be carried out in a suitable solvent such as toluene at a temperature sufficient to drive off water formed during the reaction. The reaction can also be carried out in a solvent such as ethanol or methanol in the presence of a base such as sodium hydroxide. Some compounds of Formula II are known; see for example, International Publication Numbers WO2003/011864 (DiCesare) and WO2005/026164 (Brehm) and the references cited therein.

In step (4) of the Reaction Scheme, the chloro group in a 4-chloro-1H-imidazo[4,5-c]pyridine of Formula II is displaced With a benzylamine of Formula III, as defined above, to provide an N-benzyl-1H-imidazo[4,5-c]pyridin-4-amine of Formula IV.

In some embodiments, the reaction is carried out by combining a compound of Formula II, a benzylamine of Formula III, an acid having a pKa≦9.3, and a polar solvent. In some embodiments, at least 1 equivalent, relative to the amount of compound of Formula II, of the benzylamine is used. In other embodiments, about 2 equivalents of the benzylamine are used. In other embodiments, about 5 equivalents of the benzylamine are used. In some embodiments the acid has a pKa≦5. In some embodiments the acid is selected from the group consisting of pyridine hydrochloride, hydrochloric acid, ammonium chloride, acetic acid, pyridinium tosylate, and trifluoroacetic acid. In some of these embodiments the acid used is pyridine hydrochloride. In some embodiments, a catalytic amount of the acid is used. In some embodiments, the number of equivalents of acid used is less than or equal to the number of equivalents of the benzylamine that are being used. In some of these embodiments, the molar ratio of the acid to the benzylamine is 2:5. In some embodiments, the polar solvent is a protic solvent. In some of these embodiments, the solvent is selected from the group consisting of methanol, ethanol, 1-propanol, 2-propanol, 1-butanol, 2-butanol, 2-methyl-1-propanol, 2-methyl-2-propanol, 2,2,2-trifluoroethanol, ammonium hydroxide, water, and mixtures thereof. In some of these embodiments, the solvent is 2,2,2-trifluoroethanol. In some embodiments, the polar solvent is an aprotic solvent. In some of these embodiments, the solvent is selected from the group consisting of acetonitrile, acetone, 1-methyl-2-pyrrolidinone, dimethyl sulfoxide, pyridine, N,N-dimethylformamide, 1,3-dimethyl-3,4,5,6-tetrahydro-2(1H)-pyrimidinone, and mixtures thereof.

In some embodiments, the reaction is carried out at ambient pressure. In some of these embodiments the reaction is carried out at an elevated temperature. In some of these embodiments, the reaction is carried out at the reflux temperature of the polar solvent. In other of these embodiments, the reaction is carried out at a temperature not lower than 60° C.

In some embodiments, the reaction is carried out in a pressure vessel. In some of these embodiments the reaction is carried out at an elevated temperature. In some of these embodiments, the reaction is carried out at a temperature of at least about 60° C. In some of these embodiments, the reaction is carried out at a temperature of at least about 120° C. In other of these embodiments, the reaction is carried out at a temperature less than about 250° C. In other of these embodiments, the reaction is carried out at a temperature less than about 180° C. In some embodiments, the reaction is carried out at about 150° C.

In some embodiments, the reaction is carried out in a microwave.

In step (5) of the Reaction Scheme, the benzyl group of an N-benzyl-1H-imidazo[4,5-c]pyridin-4-amine of Formula IV is removed to provide a 1H-imidazo[4,5-c]pyridin-4-amine of Formula I. For some embodiments, the benzyl group can be removed by solvolysis under acidic conditions. For example, an N-benzyl-1H-imidazo[4,5-c]pyridin-4-amine of Formula IV can be dissolved in trifluoroacetic acid and stirred at ambient temperature. In other embodiments, the reaction can be carried out on a Parr apparatus under hydrogenolysis conditions using a suitable heterogeneous catalyst such as palladium on carbon in a solvent such as ethanol.

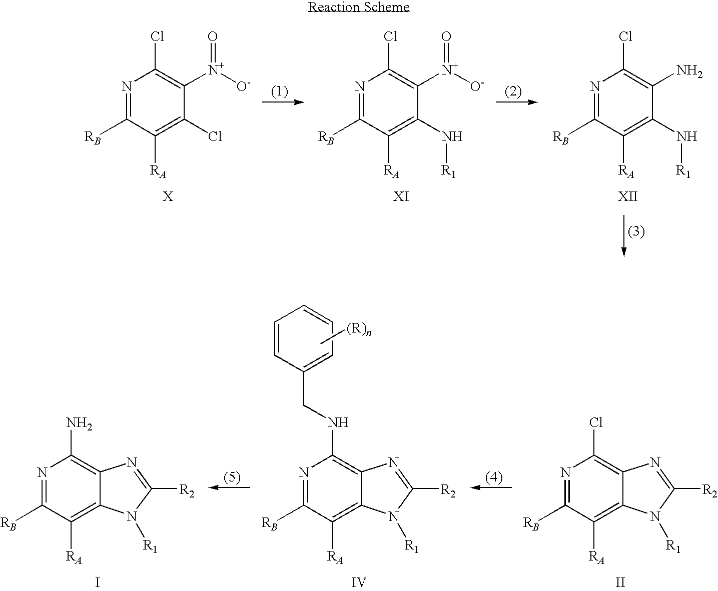

For some embodiments, compounds of Formula I can be further elaborated using conventional synthetic methods. Amines of Formula R₁—NH₂ may contain a protected functional group, such as a tert-butoxycarbonyl-protected amino group. For example, protected diamines of Formula Boc—N(R₈)—X—NH₂,

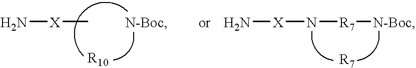

are commercially available or can be prepared by known methods; see, for example, U.S. Pat. No. 6,660,747 (Crooks), U.S. Pat. No. 6,683,088 (Crooks), and U.S. Pat. No. 6,656,938 (Crooks) and Carceller, E. et al., J. Med Chem., 39, pp.487-493 (1996). The protecting group may be removed after the cyclization step shown in step (3) of the Reaction Scheme or after one of the later steps to reveal, for example, an amino substituent on the R₁ group. An amino group introduced in this manner can react with an acid chloride of Formula R₄C(O)Cl, a sulfonyl chloride of Formula R₄S(O)₂Cl, a sulfonic anhydride of Formula (R₄S(O)₂)₂O, or an isocyanate of Formula R₄N═C═O to provide a compound of Formula I in which R₁ is —X—N(R₈)-Q-R₄,

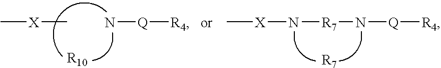

where X, R₄, R₇, R₈, and R₁₀ are as defined above and Q is —C(O)—, —SO₂—, or —C(O)—NH—. Numerous acid chlorides, sulfonyl chlorides, sulfonic anhydrides, and isocyanates are commercially available; others can be readily prepared using known synthetic methods. The reaction can be carried out by combining the acid chloride, sulfonyl chloride, sulfonic anhydride, or isocyanate and a solution of an amino-substituted compound, and a base such as triethylamine in a suitable solvent such as dichloromethane. The reaction can be carried out at room temperature.

Amines of formula R₁NH₂ can also contain other protected functional groups, such as ketal-protected ketones. For example, 2,2-dimethyl-3-(2-methyl-1,3-dioxolan-2-yl)propylamine, prepared in Example 22 of International Patent Application Publication Nos. WO2005/051317 (Krepski), can be used in step (1) of the Reaction Scheme. The ketal protecting group can later be removed by conventional methods to provide a compound of Formula I in which R₁ is 2,2-dimethyl-4-oxopentyl.

Amino alcohols of Formula H₂N—X—OH can be used in step (1) of the Reaction Scheme, and the hydroxy functional group can be converted in subsequent steps to a compound of Formula I having an —X—S(O)₀₋₂—R₄, —X—S(O)₂—N(R₈)—R₄, —X—O—N(R₈)-Q-R₄, —X—O—N═C(R₄)—R₄, or —X—CH(—N(—O—R₈)-Q-R₄)—R₄ group at the R₁ position using methods described in U.S. Pat. No. 6,664,264 (Dellaria) and International Patent Application Publication Nos. WO2005/066169 (Bonk), WO2005/018551 (Kshirsagar), WO2005/018556 (Kshirsagar), and WO2005/051324 (Krepski), respectively.

Other transformations at the R₁ position can also be made. See, for example, U.S. Pat. No. 5,389,640 (Gerster), U.S. Pat. No. 6,331,539 (Crooks), U.S. Pat. No. 6,451,810 (Coleman), U.S. Pat. No. 6,541,485 (Crooks), U.S. Pat. No. 6,660,747 (Crooks), U.S. Pat. No. 6,670,372 (Charles), U.S. Pat. No. 6,683,088 (Crooks), U.S. Pat. No. 6,656,938 (Crooks), U.S. Pat. No. 6,664,264 (Dellaria), U.S. Pat. No. 6,677,349 (Griesgraber), and U.S. Pat. No. 6,664,260 (Charles).

For some embodiments, synthetic transformations can be made at the R₂ position in a compound of Formula I, if, for example, the carboxylic equivalent used in step (3) of the Reaction Scheme contains a protected or unprotected hydroxy group or a protected amino group. Some carboxylic acid equivalents of this type are commercially available; others can be prepared by known synthetic methods. A protected hydroxy or amino group installed at the R₂ position can be deprotected by a variety of methods well known to one of skill in the art. For example, a hydroxyalkylenyl group is conveniently introduced at the R₂ position by the dealkylation of a methoxy- or ethoxyalkylenyl group, which can be installed by using a methoxy- or ethoxy-substituted carboxylic acid equivalent in step (3) of the Reaction Scheme. The dealkylation can be carried out by treating a compound of Formula I wherein R₂ is an alkoxyalkylenyl group with boron tribromide in a suitable solvent such as dichloromethane at a sub-ambient temperature such as 0° C. The resulting hydroxy group may then be oxidized to an aldehyde or carboxylic acid or converted to a leaving group such as, for example, a chloro group using thionyl chloride or a trifluoromethanesulfonate group using trifluoromethanesulfonic anhydride. The resulting leaving group can then be displaced by a variety of nucleophiles. Sodium azide can be used as the nucleophile to install an azide group, which can then be reduced to an amino group using heterogeneous hydrogenation conditions. An amino group at the R₂ position can be converted to an amide, sulfonamide, sulfamide, or urea using conventional methods. A leaving group at R₂, such as a chloro or trifluoromethanesulfonate group, can also be displaced with a secondary amine, a substituted phenol, or a mercaptan in the presence of a base such as potassium carbonate. For examples of these and other methods used to install a variety of groups at the R₂ position, see U.S. Pat. No. 5,389,640 (Gerster).

### Examples

Objects and advantages of this invention are further illustrated by the following examples, but the particular materials and amounts thereof recited in these examples, as well as other conditions and details, should not be construed to unduly limit this invention.

In the examples below normal high performance flash chromatography (prep HPLC) was carried out using a COMBIFLASH system (an automated high-performance flash purification product available from Teledyne Isco, Inc., Lincoln, Nebr., USA), a HORIZON HPFC system (an automated high-performance flash purification product available from Biotage, Inc, Charlottesville, Va., USA) or an INTELLIFLASH Flash Chromatography System (an automated flash purification system available from AnaLogix, Inc, Burlington, Wis., USA). The eluent used for each purification is given in the example. In some chromatographic separations, the solvent mixture 80/18/2 v/v/v chloroform/methanol/concentrated ammonium hydroxide (CMA) was used as the polar component of the eluent. In these separations, CMA was mixed with chloroform in the indicated ratio.

### Example 1

### (4-Amino-1-isobutyl-6,7-dimethyl-1H-imidazo[4,5-c]pyridin-2-yl)methanol

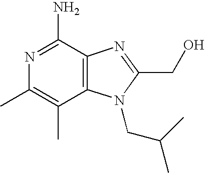

### Part A

A solution of 2,4-dichloro-5,6-dimethyl-3-nitropyridine (40.0 g, 181 mmol), triethylamine (26.5 mL, 190 mmol), and isobutyl amine (18.9 mL, 190 mmol) in N,N-dimethylformamide (500 mL) was stirred at room temperature over night. The solvent was removed under reduced pressure. The residue was dissolved in ethyl acetate (500 mL) and washed with water (3×80 mL) and brine (40 mL). The aqueous was extracted with ethyl acetate (3×50 mL) and the back-extracts washed with water (3×40 mL) and brine (30 mL). The combined organics were dried over magnesium sulfate, filtered, and concentrated under reduced pressure. The crude product was purified by prep HPLC eluting with a gradient of 10-30% ethyl acetate in hexanes to give 25.8 g of 2-chloro-N-isobutyl-5,6-dimethyl-3-nitropyridin-4-amine as a yellow oil.

### Part B

2-Chloro-N-isobutyl-5,6-dimethyl-3-nitropyridin-4-amine (25.8 g, 100 mmol) was combined with 5% platinum on carbon (2.58 g) and ethyl acetate (200 mL) in a pressure vessel and hydrogenated at 50 psi (3.4×10⁵ Pa) for 2.5 hours on a Parr apparatus. The reaction mixture was filtered through CELITE filter agent, which was rinsed with ethyl acetate and methanol afterwards. The filtrate was concentrated to give 2-chloro-N⁴-isobutyl-5,6-dimethylpyridine-3,4-diamine and was used directly in the next step.

### Part C

Under a nitrogen atmosphere, the material from part B was dissolved in dichloromethane (400 mL) and cooled to 0° C. Ethoxyacetyl chloride (14.7 g, 120 mmol) dissolved in dichloromethane (100 mL) was added dropwise through an addition funnel and the solution was stirred at room temperature over night. The solvent was removed under reduced pressure and the white solid used directly in the next step.

### Part D

The material from part C was suspended in ethanol (500 mL), and sodium hydroxide (10.0 g, 250 mmol) was added. The mixture was heated to reflux under a nitrogen atmosphere for 4 hours. The heat was removed and the solution allowed to stir at room temperature over night. The solvent was removed under reduced pressure and the residue was dissolved in dichloromethane (500 mL), washed with water (100 mL) and brine (60 mL), dried over sodium sulfate, filtered, and concentrated under reduced pressure to give 29.6 g of 4-chloro-2-(ethoxymethyl)-1-isobutyl-6,7-dimethyl-1H-imidazo[4,5-c]pyridine as a yellow oil.

### Part E

A 500 mL round bottom flask was charged with 4-chloro-2-(ethoxymethyl)-1-isobutyl-6,7-dimethyl-1H-imidazo[4,5-c]pyridine (10.0 g, 33.8 mmol) and dichloromethane (250 mL) under a nitrogen atmosphere. The solution was cooled to 0° C. and boron tribromide (101 mL of 1M in dichloromethane, 101 mmol) was added through an addition funnel over 30 minutes. The reaction mixture was allowed to warm to room temperature and was stirred over night. Methanol was added slowly until no more fizzing occurred, then the solvent was partially removed under reduced pressure. More methanol was added (100 mL) as well as 6N hydrochloric acid (100 mL) and the solution was heated at reflux for 1 hour. The reaction mixture was then allowed to cool to room temperature and stirred over night. The solvent was partially removed under reduced pressure until a solid precipitated, which was filtered and washed with water, then triturated with ethyl acetate and hexanes to give 6.15 g of (4-chloro-l-isobutyl-6,7-dimethyl-1H-imidazo[4,5-c]pyridin-2-yl)methanol as an off-white solid.

### Part F

A solution of (4-chloro-1-isobutyl-6,7-dimethyl-1H-imidazo[4,5-c]pyridin-2-yl)methanol (1.50 g, 5.60 mmol), 4-methoxybenzylamine (3.66 mL, 28.0 mmol), and pyridine hydrochloride (1.74 g, 11.2 mmol) in 2,2,2-trifluoroethanol (11.2 mL) was heated to 150° C. in a microwave oven for 2.5 hours. The mixture was allowed to cool to room temperature, then poured into water (75 mL) and stirred for 30 minutes. The precipitate was filtered off and washed with water to give 1.86 g of {1-isobutyl-4-[(4-methoxybenzyl)amino]-6,7-dimethyl-1H-imidazo[4,5-c]pyridin-2-yl}methanol.

### Part G

{1-Isobutyl-4-[(4-methoxybenzyl)amino]-6,7-dimethyl-1H-imidazo[4,5-c]pyridin-2-yl}methanol (1.00 g, 2.71 mmol) was dissolved in trifluoroacetic acid (15 mL) and stirred at room temperature over night. The solvent was removed under reduced pressure and concentrated hydrochloric acid (5 mL) was added. The suspension was stirred for 3 hours, then 6N sodium hydroxide was added until a precipitate formed. The mixture was filtered and the filter cake washed with water. The filtrate was extracted with a mixture of chloroform and methanol, the organic extracts were combined with the filter cake from above and concentrated in vacuo to give a white solid. The crude product was triturated twice with acetonitrile, purified by prep HPLC eluting with a gradient of 0-40% CMA in chloroform and then triturated with acetonitrile to provide 505 mg of (4-amino-1-isobutyl-6,7-dimethyl-1H-imidazo[4,5-c]pyridin-2-yl)methanol as a white powder, mp 231.0-234.0° C. Anal. Calcd for C₁₃H₂₀N₄O C, 62.88; H, 8.12; N, 22.56; Found: C, 62.95; H, 7.84; N, 22.66.

### Example 2

### 2-(Ethoxymethyl)-1-isobutyl-6,7-dimethyl-1H-imidazo[4,5-c]pyridin-4-amine

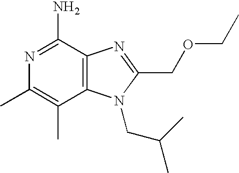

### Part A

A solution of 4-chloro-2-(ethoxymethyl)-1-isobutyl-6,7-dimethyl-1H-imidazo [4,5-c]pyridine (1.66 g, 5.60 mmol, prepared as in parts A-D of example 1), 4-methoxybenzylamine (3.66 mL, 28.0 mmol), and pyridine hydrochloride (1.74 g, 11.2 mmol) in 2,2,2-trifluoroethanol (11.2 mL) was heated to 150° C. in a microwave oven for 2.5 hours. More 4-methoxybenzylamine (1.0 mL, 7.7 mmol) was added and the solution was heated to 150° C. in a microwave oven for another 30 minutes. The mixture was allowed to cool to room temperature, then poured into water (75 mL) and extracted with dichloromethane (3×70 mL). The combined organics were washed with 5% w/v aqueous citric acid (4×30 mL), saturated aqueous sodium bicarbonate (30 mL) and brine (20 mL), dried over magnesium sulfate, filtered, and concentrated under reduced pressure to give 2.10 g of 2-(ethoxymethyl)-1-isobutyl-N-(4-methoxybenzyl)-6,7-dimethyl-1H-imidazo[4,5-c]pyridin-4-amine.

### Part B

The material from part A was dissolved in trifluoroacetic acid (21 mL) and stirred at room temperature over night. The solvent was removed under reduced pressure and concentrated hydrochloric acid (10 mL) was added. The suspension was stirred for 3 hours, then 6N sodium hydroxide was added until the pH was basic. The solution was extracted with dichloromethane (3×80 mL) and the combined organics were washed with saturated aqueous sodium chloride (30 mL), dried over sodium sulfate, filtered, and concentrated under reduced pressure. The crude product was purified by prep HPLC eluting with a gradient of 0-30% CMA in chloroform and then triturated with acetonitrile to provide 87 mg of 2-(ethoxymethyl)-1-isobutyl-6,7-dimethyl-1H-imidazo[4,5-c]pyridin-4-amine as a white powder, mp 110.0-112.0° C. Anal. Calcd for C₁₅H₂₄N₄OC, 65.19; H, 8.75; N, 20.27; Found: C, 65.16; H; 8.37; N, 20.38.

### Example 3

Three separate microwave vessels (0.5-2 mL) were charged as shown in Table 1 below where starting material is 4-chloro-2-(ethoxymethyl)-1-isobutyl-6,7-dimethyl-1H-imidazo[4,5-c]pyridine. The vessels were heated in a microwave at 150° C. for 30 minutes. The reaction mixtures were analyzed for the presence of both starting material and the desired product (2-(ethoxymethyl)-1-isobutyl-N-(4-methoxybenzyl)-6,7-dimethyl-1H-imidazo[4,5-c]pyridin-4-amine) by high performance liquid chromatography (Hewlett-Packard model 1100 series HPLC system equipped with a 4.6×50 mm MICROSORB-MV C-18 column; flow rate: 1 mL/minute; solvent gradient: 99% water containing 0.1% trifluoroacetic acid, 1% acetonitrile containing 0.1% trifluoroacetic acid to 1% water containing 0.1% trifluoroacetic acid, 99% acetonitrile containing 0.1% trifluoroacetic acid over 5 minutes; hold for 2 minutes; return to initial gradient conditions; injection volume: 10 μL; detector: diode array at 254 nm with a 450 nm reference). The results are shown in Table 1 below.

TABLE 1

Vessel

| Reagent | A | B | C |
|---|---|---|---|
| Starting material | 100 mg | 100 mg | 100 mg |
| 4-Methoxybenzylamine | 221 μL | 221 μL | 221 μL |
| 2,2,2-Trifluoroethanol | 680 μL | 680 μL | 680 μL |
| Pyridine hydrochloride | None | 105 mg | None |
| 4N HCl in dioxane | None | None | 169 μL |
| % Starting material | 100 | 45 | 52 |
| % Product | 0 | 55 | 48 |

### Example 4

Three separate vials (4 mL) were charged as shown in Table 2 below where starting material is 4-chloro-2-(ethoxymethyl)-1-isobutyl-6,7-dimethyl-1H-imidazo[4,5-c]pyridine. The vials were sealed with TEFLON and then heated in a pressure vessel at 150° C. for 20 hours. The reaction mixtures were analyzed for the presence of both starting material and the desired product (2-(ethoxymethyl)-1-isobutyl-N-(4-methoxybenzyl)-6,7-dimethyl-1H-imidazo[4,5-c]pyridin-4-amine) by high performance liquid chromatography as described in Example 3. The results are shown in Table 2 below.

TABLE 2

Vial

| Reagent | A | B | C |
|---|---|---|---|
| Starting material | 71 mg | 71 mg | 71 mg |
| 4-Methoxybenzylamine | 158 μL | 158 μL | 158 μL |
| 2,2,2-Trifluoroethanol | 480 μL | 480 μL | 480 μL |
| Pyridine hydrochloride | None | 75 mg | None |
| 4N HCl in dioxane | None | None | 121 μL |
| % Starting material | 56 | 0 | 0 |
| % Product | 44 | 100 | 100 |

### Example 5

The experiment described in Example 4 was repeated except that the reaction mixtures were heated for 4 hours instead of 20 hours. The results are shown in Table 3 below.

TABLE 3

Vial

| Reagent | A | B | C |
|---|---|---|---|
| Starting material | 71 mg | 71 mg | 71 mg |
| 4-Methoxybenzylamine | 158 μL | 158 μL | 158 μL |
| 2,2,2-Trifluoroethanol | 480 μL | 480 μL | 480 μL |
| Pyridine hydrochloride | None | 75 mg | None |
| 4N HCl in dioxane | None | None | 121 μL |
| % Starting material | 99 | 30 | 36 |
| % Product | 1 | 70 | 64 |

We claim:

1. A method for preparing a 1H-imidazo[4,5-c]pyridine compound or a pharmaceutically acceptable salt thereof comprising:
combining a compound of the Formula II: 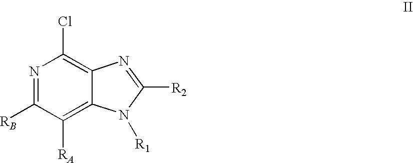
with a benzylamine of the Formula III: 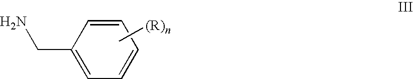
in the presence of an acid to provide a 1H-imidazo[4,5-c]pyridine compound of the Formula IV: 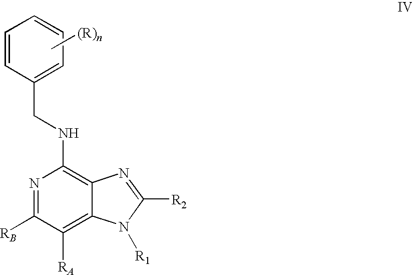
or a pharmaceutically acceptable salt thereof;
wherein in the above Formulas III and IV:
each R is independently selected from the group consisting of alkyl, alkoxy, aryloxy, alkylamino, dialkylamino, arylamino, alkylthio, arylthio, and halogen; and n is 0, 1, 2, or 3; and
wherein in the above Formulas II and IV:
R_{A} and R_{B} are independently selected from the group consisting of:
hydrogen,
halogen,
alkyl,
alkenyl,
alkoxy,
alkylthio, and
—N(R₉)₂;
R₁ is selected from the group consisting of:
—R₄,
—X—R₄,
—X—Y—R₄,
—X—Y—X—Y—R₄, and
—X—R₅,
R₂ is selected from the group consisting of:
—R₄,
—X—R₄,
—X—Y—R₄, and
—X—R₅;
X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;
Y is selected from the group consisting of:
—O—,
—S(O)₀₋₂—,
—S(O)₂—N(R₈)—,
—C(R₆)—,
—C(R₆)O—,
—O—C(R₆)—,
—0—C(O)—O—,
—N(Rs)-Q-,
—C(Re)—N(R₈)—,
—O—C(R₆)—N(R₈)—,
—C(R₆)—N(OR₈)—,
—O—N═C(R₄)—,
—CH(—N(—O—R₈)-Q-R4)—, 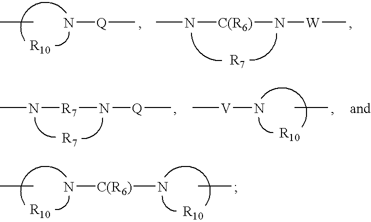
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, heterocyclyl, and heterocyclylalkylenyl; wherein the alkyl, alkenyl alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo; and wherein the heterocyclylalkylenyl group is optionally substituted by one or more alkyl groups
R₅ is selected from the group consisting of: 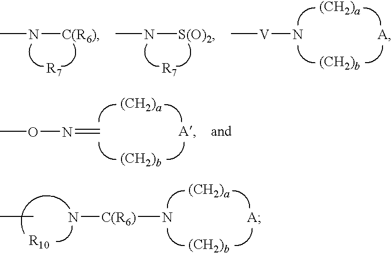
R₆ is selected from the group consisting of ═O and ═S;
R₇ is C₂₋₇ alkylene;
R₈ is selected from the group consisting of hydrogen, alkyl, alkoxyalkylenyl, hydroxyalkylenyl, arylalkylenyl, and heteroarylalkylenyl;
R₉ is selected from the group consisting of hydrogen and alkyl;
R₁₀ is C₃₋₈ alkylene;
A is selected from the group consisting of —CH₂—, —O—, —C(O)—, —S(O)₀₋₂— and —N(Q-R₄)—;
A¹ is selected from the group consisting of —O—, —S(O)₀₋₂—, —N(-Q-R₄)—, and —CH₂—;
Q is selected from the group consisting of a bond, —C(R₆)—, —C(R₆)—C(R₆)—, —S(O)₂—; —C(R₆)—N(R₈)—W—, —S(O)₂—N(R₈)—, —C(R₆)—O—, —C(R₆)—S—, and —C(R₆)—N(OR₉)—;
V is selected from the group consisting of —C(R₆)—, —O—C(R₆)—, —N(R₈)—C(R₆)—, and —S(O)₂—;
W is selected from the group consisting of a bond, —C(O)—, and —S(O)₂—; and a and b are independently integers from 1 to 6 with the proviso that a+b is ≦7; with the proviso that R₁ is other than (4-hydroxytetrahydro-2H-pyran-4-yl)methyl.

2. The method of claim 1 wherein the compound of Formula II is combined with at least one equivalent of the compound of Formula III.

3. The method of claim 2 wherein the compound of Formula II is combined with up to five equivalents of the compound of Formula III.

4. The method of claim 2, wherein the compound of Formula III is selected from the group consisting of benzylamine, 4-methoxybenzylamine, 2,4-dimethoxybenzylamine, and 3,4-dimethoxybenzylamine.

5. The method of claim 1 wherein the pKa of the acid is up to 9.3.

6. The method of claim 5 wherein the pKa of the acid is up to 5.

7. The method of claim 1 wherein the number of equivalents of acid present is equal to or less than the number of equivalents of the compound of Formula III.

8. The method of claim 1 wherein a solvent is present.

9. The method of claim 8 wherein the solvent is less nucleophilic than the compound of Formula III.

10. The method of claim 9 wherein the solvent is a polar solvent.

11. The method of claim 10 wherein the solvent is 2,2,2-trifluoroethanol.

12. The method of claim 1 wherein the temperature of the combined compounds in the presence of the acid is at least room temperature.

13. The method of claim 12 wherein the temperature is up to 250° C.

14. The method of claim 13 wherein the temperature is up to 180° C.

15. The method of claim 1 wherein the temperature of the combined compounds in the presence of the acid is provided by exposure to microwaves.

16. The method of claim 1 wherein the temperature of the combined compounds in the presence of the acid is provided by external heating.

17. The method of claim 1 further comprising a step selected from the group consisting of solvolyzing the compound or salt of Formula IV and hydrogenolyzing the compound or salt of Formula IV to provide a 1H-imidazo[4,5-c]pyridin-4-amine compound of the Formula I: 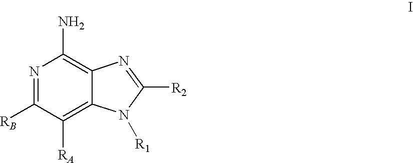
or a pharmaceutically acceptable salt thereof, wherein R₁, R₂, R_{A}, and R_{B} are defined as in claim 1.

18. The method of claim 17, further comprising the step of isolating the compound of Formula I or a pharmaceutically acceptable salt thereof.

19. The method of claim 1 further comprising the steps of providing a compound of the Formula XII: 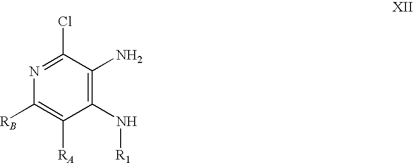
wherein R_{A}, R_{B} and R₁ are defined as in claim 1;
and reacting the compound of the Formula XII with a carboxylic acid of the formula R₂CO₂H; an equivalent thereof selected from the corresponding acyl halide, R₂C(O-alkyl)₃, and R₂C(O-alkyl)₂(O—C(O)-alkyl); or a mixture thereof, wherein R₂ is defined as in claim 1, and each alkyl contains 1 to 8 carbon atoms, to provide a compound of the Formula II: 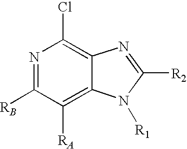
wherein R_{A}, R_{B}, R₁, and R₂ are defined as in claim 1.

20. The method of claim 19 further comprising the steps of providing a compound of the Formula XI: 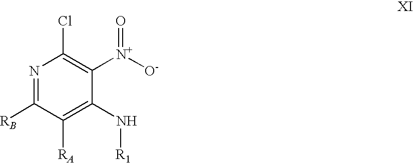
wherein R_{A}, R_{B}, and R₁ are defined as in claim 1;
and reducing the compound of Formula XI to provide a compound of the Formula XII.

21. The method of claim 20 further comprising the steps of providing a compound of the Formula X: 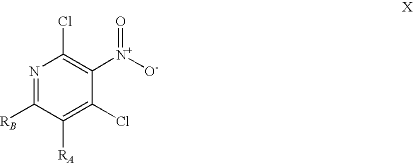
X wherein R_{A} and R_{B} are defined as in claim 1;
and reacting the compound of Formula X with an amine of Formula R₁—NH₂, wherein R₁ is defined as in claim 1, to provide a compound of Formula XI.

22. The method of claim 1 wherein R_{A} is hydrogen or alkyl, and R_{B} is alkyl.

23. The method of claim 22 wherein R_{A} and R_{B} are both methyl.

24. The method of claim 1 wherein R₁ is selected from the group consisting of alkyl, arylalkylenyl, aryloxyalkylenyl, hydroxyalkyl, dihydroxyalkyl, alkoxyalkylenyl, alkylsulfonylalkylenyl, —X—Y—R₄—X—R₅, and heterocyclylalkylenyl; wherein the heterocyclyl of the heterocyclylalkylenyl group is optionally substituted by one or more alkyl groups; wherein X is alkylene; Y is —N(R₈)—C(R₆)—, —N(R₈)—S(O)₂—, —N(R₈)—C(O)—N(R₈)—, —C(R₆)—N(R₈)—, —C(R₆)—O—, or 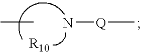
R₄ is alkyl, aryl, or heteroaryl; and R₅ is

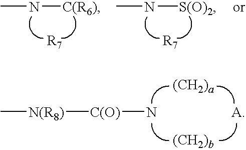

25. The method of claim 24 wherein R₁ is selected from the group consisting of 2-hydroxy-2-methylpropyl, 2-methylpropyl, propyl, ethyl, methyl, 2,3-dihydroxypropyl, 3-isopropoxypropyl, 2-phenoxyethyl, 4-[(methylsulfonyl)amino]butyl, 2-methyl-2-[(methylsulfonyl)amino]propyl, 2-(acetylamino)-2-methylpropyl, 2-{[(isopropylamino)carbonyl]amino}-2-methylpropyl,4-{[(isopropylamino)carbonyl]amino}butyl, 4-(1,1-dioxidoisothiazolidin-2-yl)butyl, tetrahydro-2H-pyran-4-ylmethyl, and (2,2-dimethyl-1,3-dioxolan-4-yl)methyl.

26. The method of claim 1 wherein R₂ is selected from the group consisting of hydrogen, alkyl, alkoxyalkylenyl, and hydroxyalkylenyl.

27. The method of claim 26 wherein R₂ is selected from the group consisting of hydrogen, methyl, ethyl, propyl, butyl, ethoxymethyl, methoxymethyl, 2-methoxy ethyl, hydroxy methyl, and 2-hydroxyethyl.
